(19) United States
(12) Patent Application Publication
(10) Pub. No.: US 2008/0269844 A1
(43) Pub. Date: Oct. 30, 2008

(54) Reflective Crib Liner

(76) Inventor: Kimberly D. Logslett, Colfax, WI (US)

Correspondence Address: MOORE & HANSEN, PLLP, 225 SOUTH SIXTH ST, MINNEAPOLIS, MN, 55402, US

(21) Appl. No.: 11/742,071

(22) Filed: Apr. 30, 2007

(51) Int. Cl. A61N 5/06; A47C 31/00

(52) U.S. Cl. 607/88; 5/663

(57) ABSTRACT

A reflective crib liner for improving the efficacy of phototherapy is disclosed. The reflective crib liner has a fabric layer having dimensions to accommodate placement of the reflective crib liner in a bassinet used in a phototherapy treatment. The bassinet has at least one wall that defines an interior surface. A reflective layer is formed so as to cover at least a portion of the interior surface. The reflective layer has a first degree of reflectance with respect to visible light and a second degree of reflectance with respect to heat that is substantially lower than the first degree of reflectance. The reflective layer comprises a porous material having a light diffusing surface.

### TECHNICAL BACKGROUND

The disclosure relates generally to phototherapy treatment techniques. More particularly, the disclosure relates to phototherapy treatment of hyperbilirubinemia in infants.

### BACKGROUND

Hyperbilirubinemia is a condition in which serum bilirubin levels in a person's blood stream rise above certain unacceptable levels. Generally, this is readily noticeable as high levels of serum bilirubin are typically characterized by jaundice, a yellow or orange discoloration of the affected person's skin. While jaundice is a common condition among newborn infants, and often resolves itself without any treatment or ill effect, high levels of serum bilirubin can adversely affect the brain and nervous system of a newborn. Accordingly, where a clinically significant case of hyperbilirubinemia is identified, it is imperative to lower the child's level of serum bilirubin as soon as is possible.

In extreme cases, hyperbilirubinemia is treated by transfusing the patient. Less acute cases have generally been treated using phototherapy. In order to be removed from the body, serum bilirubin must be modified so that it can be filtered from the blood stream by the liver and excreted into the bowel. Because serum bilirubin is present in the skin, and may be converted into a nontoxic isomer by exposure to blue or cool white light, phototherapy, a noninvasive treatment requiring only the application of lights to the patient's skin is preferred.

In phototherapy, light is shone on the skin of the patient in order to break down the serum bilirubin into its isomer lumirubin, which is diffused into the blood and subsequently bound to albumin. Thereafter, the albumin-bound lumirubin is transported to the liver and excreted as indicated above. The chemical structure of the serum bilirubin is such that light having a wavelength in the range of approximately 410 to 500 nanometers will induce the isomerization of bilirubin into lumirubin. It is recognized that the conversion of serum bilirubin into a nontoxic isomer such as lumirubin is the limiting factor in reducing serum bilirubin levels. Accordingly, it is desirable to apply as much light in the range of wavelengths given above, hereinafter referred to as therapeutic light, as is possible.

One method used to increase the intensity of therapeutic light incident upon a patient is to provide a reflector within the bassinet within which the newborn is laid. A common practice is to line the interior of the bassinet with common aluminum foil so as to reflect as much therapeutic light onto the child as is possible. Unfortunately, the use of aluminum foil as a reflector also acts to raise the body temperature of the child, as the foil will reflect more infrared radiation onto the child's body. Because a child's body temperature cannot be allowed to rise above a certain level, phototherapy treatments must often be cut short in order to allow the child to cool down. Another drawback to the use of aluminum foil as a reflector in phototherapy is that the appearance of the aluminum foil can be somewhat disconcerting to some parents. The aluminum foil has no aesthetic appeal, and if a parent remarks on it at all, it is typically in a negative fashion.

### SUMMARY OF THE DISCLOSURE

According to various example embodiments, a liner for a bassinet or crib can be used in conjunction with phototherapy. The liner has a relatively high reflectance with respect to therapeutic light, but a relatively low reflectance with respect to infrared radiation.

One embodiment is directed to a reflective crib liner for improving the efficacy of a phototherapy session performed using a bassinet. The reflective crib liner has a fabric layer having dimensions to accommodate placement of the reflective crib liner in the bassinet. The bassinet has at least one wall that defines an interior surface having a bottom portion. A reflective layer is formed so as to cover at least a portion of the interior surface. The reflective layer has a first degree of reflectance with respect to visible light and a second degree of reflectance with respect to heat that is substantially lower than the first degree of reflectance. The reflective layer comprises a porous material having a light diffusing surface.

In another embodiment, a phototherapy system includes a bassinet comprising at least one wall defining an interior surface having a bottom portion and a phototherapy light source mounted over the bassinet. A reflective crib liner is located in the bassinet. The reflective crib liner has a fabric layer and a reflective layer formed so as to cover at least a portion of the interior surface of the bassinet. The reflective layer has a first degree of reflectance with respect to visible light and a second degree of reflectance with respect to heat that is substantially lower than the first degree of reflectance. The reflective layer comprises a porous material having a light diffusing surface.

Various embodiments may provide certain advantages. For instance, because the reflective layer is relatively reflective of visible light, a greater amount of the light emitted by the phototherapy light source is directed toward the patient. In addition, due to the relatively low level of heat reflectivity and the porosity of the reflective layer, relatively little heat is transmitted to the patient. Accordingly, phototherapy sessions using the reflective crib liner disclosed herein may be conducted over greater durations relative to phototherapy sessions using some conventional phototherapy methods and equipment. Improvement in the efficacy of phototherapy may be realized as a result.

Additional objects, advantages, and features will become apparent from the following description and the claims that follow, considered in conjunction with the accompanying drawings.

### BRIEF DESCRIPTION OF THE DRAWINGS

FIG. 1 is a sectional view illustrating a child in a bassinet receiving phototherapy for hyperbilirubinemia.

FIG. 2 is a partial cross-sectional view of a liner of the present invention taken across section line 2-2 of FIG. 1.

### DESCRIPTION OF VARIOUS EMBODIMENTS

According to various embodiments, a reflective crib liner may improve the efficacy of phototherapy for treatment of hyperbilirubinemia in infants. The reflective crib liner has a fabric layer having dimensions to accommodate placement of the reflective crib liner in a bassinet used in a phototherapy treatment. The bassinet has at least one wall that defines an interior surface. A reflective layer is formed so as to cover at least a portion of the interior surface. The reflective layer has a first degree of reflectance with respect to visible light and a second degree of reflectance with respect to heat that is substantially lower than the first degree of reflectance. The reflective layer comprises a porous material having a light diffusing surface.

In the following description, numerous specific details are set forth in order to provide a thorough understanding of various embodiments. It will be apparent to one skilled in the art that some embodiments may be practiced without some or all of these specific details. In other instances, well known components and process steps have not been described in detail.

Although this disclosure is sufficiently detailed to enable those skilled in the art to practice the embodiments described herein, the disclosed embodiments are intended as merely illustrative and not limiting. While various embodiments have been described, certain details may be changed without departing from the spirit and scope of the present invention, which is defined by the claims.

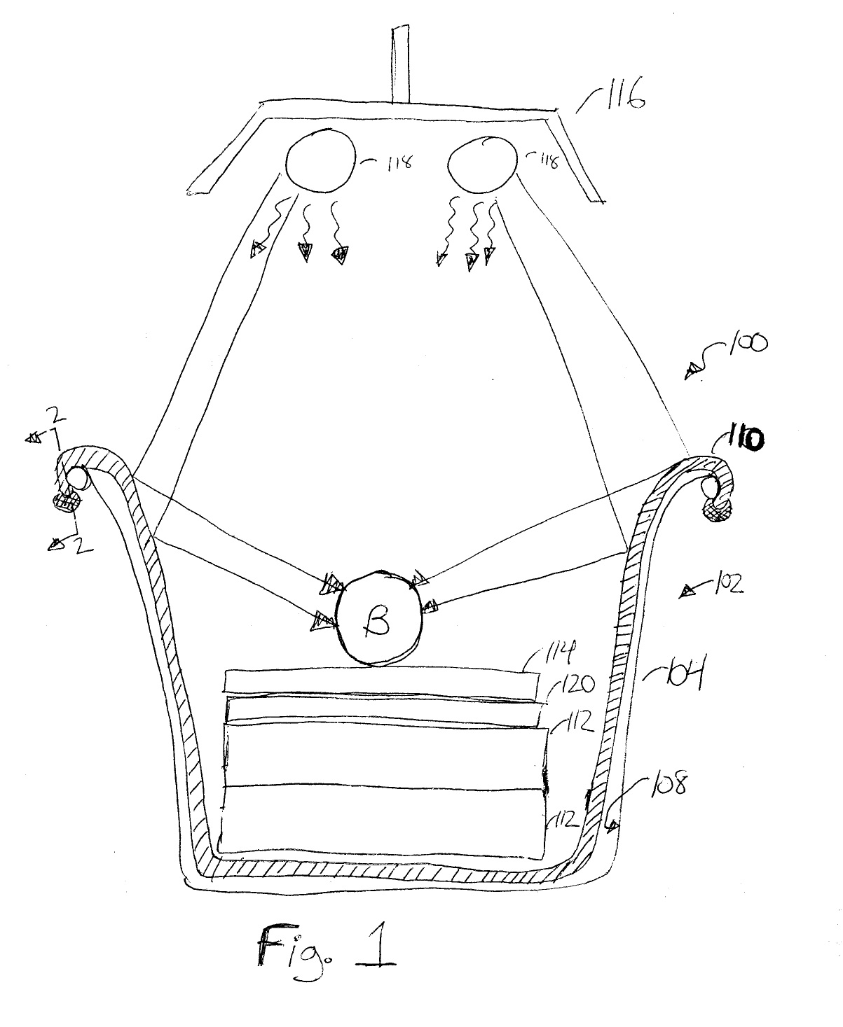

Referring now to the drawings, FIG. 1 is a sectional view illustrating a child B receiving phototherapy treatment for hyperbilirubinemia via a phototherapy system 100 according to one embodiment. The phototherapy system 100 includes a bassinet 102 that has dimensions selected to accommodate the child B. The dimensions of the bassinet 102 may conform to standard dimensions used in the industry, e.g., approximately 12″ in height. The bassinet 102 has at least one side wall 104 and a bottom wall 106 that define an interior surface 108.

A reflective crib liner 110 is located in the bassinet 102. In the embodiment depicted in FIG. 1, the reflective crib liner 110 has dimensions selected to fit in the bassinet 102 to cover at least substantially all of the side walls 104 and the bottom wall 106, i.e., most or all of the interior surface 108. In other embodiments, however, the reflective crib liner 110 may be configured to cover only the side walls 104 of the bassinet 102. One or more mattresses 112 are located in the bassinet 102 and serve to maintain the position of the reflective crib liner 110 relative to the bassinet 102. In addition, the mattresses 112 position the child B with respect to the phototherapy equipment. For example, the mattresses 112 may be arranged such that the distance from the mattresses 112 to the top of the bassinet 102 is approximately 6″. The mattresses 112 are sized to position the child B at an appropriate height relative to the phototherapy equipment. In one embodiment, for example, two mattresses 112 are used, each of which is approximately 2-3″ thick. As a result, the top surface of the top mattress 112 is located approximately 12″ from the light source.

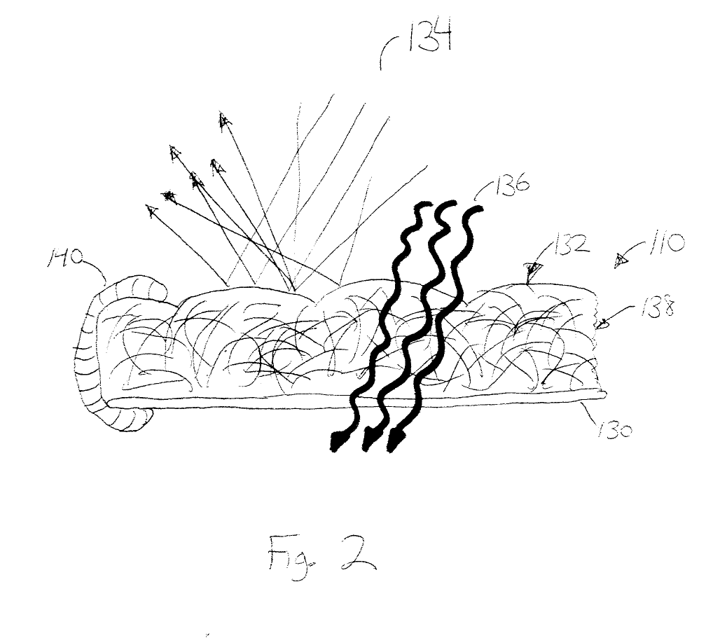

In addition to the reflective crib liner 110 and the mattresses 112, a blanket 114 may also be located in the bassinet 102, e.g., on top of a mattress 112. The child B is positioned on top of the blanket 114. The blanket 114 may have a similar construction as the reflective crib liner 110, which is described in greater detail below in connection with FIG. 2.

A phototherapy light source 116 is mounted over the bassinet. The phototherapy light source 116 may be implemented as any of a variety of phototherapy light sources known in the art. For example, the phototherapy light source 116 may employ fluorescent tubes or light emitting diodes. One suitable phototherapy light source is the Model 33 Bili-Lite™ manufactured by Olympic Medical of Seattle, Wash. The phototherapy light source 116 may employ two or more fluorescent light bulbs 118 that output light in the range of wavelengths to which serum bilirubin is sensitive. Serum bilirubin may be converted into lumirubin by the incidence of light having a wavelength of between 420 and 480 nm. Many commercially available fluorescent light bulbs 118 designed for phototherapy output light having a wavelength of between 400 and 550 nm. For instance, one particular high intensity bulb manufactured by Phillips Lighting under the part number F20T12BB emits 95% of its light intensity in the range of 425 to 475 nm. In addition to the therapeutic light in the range of 420-480 nm, the phototherapy light source 116 outputs infrared light, i.e., heat. This infrared light warms the child B and is a limiting factor in phototherapy treatments as it is desirable to prevent overheating in the child B.

The reflective crib liner 110 and, if present, the blanket 114 enhances the efficacy of phototherapy in a number of ways. First, the reflective crib liner 110 reflects light output by the phototherapy light source 116 onto the child B positioned in the bassinet 102. By reflecting light onto the child B, the intensity of the therapeutic light incident upon the child B is increased. In addition, the reflective crib liner 110 reduces heating effects on the child B by diffusing some of the infrared light that would otherwise be transmitted to the child B. Further, the reflective crib liner 110 is constructed from a porous material that allows air to circulate through the reflective crib liner 110, further reducing heating effects on the child B.

FIG. 2 illustrates an example implementation of the reflective crib liner 110. The reflective crib liner 110 includes a fabric layer 130 and a reflective layer 132 formed so as to cover at least part of the interior surface 108 of the bassinet 102 when the reflective crib liner 110 is placed in the bassinet 102. The fabric layer 130 can be constructed of any of a variety of porous fabrics, and may incorporate aesthetically appealing patterns or other designs to reduce the psychological discomfort of the parent or parents of the child B.

The reflective layer 132 has a relatively high degree of reflectance with respect to visible light, particularly visible light in the wavelength range used in phototherapy, e.g., between 420 and 480 nm. For example, the reflective layer 132 may be constructed and arranged to have a reflectance with respect to visible light of wavelengths between 420 and 480 nm that is greater than that of aluminum foil, which is commonly used as a liner in phototherapy sessions using conventional techniques and equipment. As a result, much of the light emitted by the phototherapy light source 116 is reflected toward the child B. On the other hand, the reflective layer 132 is substantially less reflective with respect to heat. For example, the reflective layer 132 may be constructed and arranged to have a reflectance with respect to heat that is less than that of aluminum foil. With the reflective layer 132 relatively non-reflective with respect to heat, relatively little of the heat emitted by the phototherapy light source 116 is reflected toward the child B.

The reflective layer 132 is formed from a porous material having a diffusing surface. With the reflective layer 132 formed in this way, light that impinges on the reflective layer 132 is diffused as it is reflected, as indicated by the solid arrows 134 of FIG. 2. Because the reflective layer 132 is porous, heat is generally transmitted through the reflective layer 132 rather than reflected toward the child B, as indicated by the wavy arrows 136 of FIG. 2. While a small amount of heat is reflected toward the child B, the amount is significantly lower than is exhibited with the use of aluminum foil, which is common in conventional phototherapy treatments. The porous nature of the reflective layer 132 facilitates air circulation, as does a layer of batting material 138 sandwiched between the reflective layer 132 and the fabric layer 130. As a result, the child B experiences significantly lower heating relative to some conventional phototherapy treatments.

In some embodiments, the reflective layer 132 is formed from a quilted material. The quilted material may have reflective metallic threads, e.g., a lamé material, or may be a pliable, reflective film. As described above, it is desirable that the reflective layer 132 be more reflective than aluminium foil with respect to visible light, but less reflective than aluminium foil with respect to heat. Alternatively, the reflective layer 132 may have different degrees of reflectivity with respect to visible light and heat, as long as the reflective layer 132 is more reflective of visible light than of heat. In one embodiment, the reflective layer is formed from a fabric comprising a metallic thread as the woof and a clear nylon thread as the warp. Fabrics of this type are commercially available, for example, from Scarsdale Quilting and Converting Corp., Inc., located in Tupelo, Miss.

The batting material 138 is a conventional non-woven batting material that has both cushioning and insulating characteristics. In use, some of the infrared light, or heat, impinging on the reflective layer 132 of the reflective crib liner 110 is reflected by the reflective layer 132. However, a significant portion of the heat is absorbed, passed, or otherwise attenuated by the reflective layer 132, batting material 138, and fabric layer 130 as shown by the wavy arrows 136. The proportion of heat that is attenuated in this manner is preferably higher for the reflective crib liner 110 than for aluminum.

In addition to reflecting visible light to the child B while attenuating heat, the reflective crib liner 110 provides a soft surface for the child B to lie upon during phototherapy treatment, thereby promoting the comfort of the child B. In particular, the batting material 138 and the fabric layer 130, which may be formed from a non-woven nylon or acrylic material, provides a soft touch and feel to the reflective crib liner 110. The reflective crib liner 110 may be used in conjunction with additional padding, such as foam, and the child B may be laid directly upon the reflective crib liner 110 without the need for a mattress 112. In addition, the reflective crib liner 110 can incorporate additional layers of insulation, padding or waterproofing, for example, between the reflective layer 132 and the fabric layer 130, or on the fabric layer 130 itself.

The reflective crib liner 110 is versatile. For example, while the reflective crib liner 110 is primarily used in phototherapy, it is sufficiently comfortable to be used as an everyday bassinet liner, even when the child B is not receiving phototherapy. In addition, while the reflective crib liner 110 is generally sized for use with a bassinet 102 having substantially standard dimensions, elastic material 140 facilitates accommodating bassinets 102 of various dimensions. The reflective crib liner 110 may also be used in conjunction with a light emitting pad such as the Bili-Lite Pad™, a fiber-optic pad sold by Olympic Medical of Seattle, Wash. and illustrated at reference numeral 120 in FIG. 1. As depicted in FIG. 1, the light emitting pad 120 is positioned under the child B.

As demonstrated by the foregoing discussion, various embodiments may provide certain advantages, particularly in the context of phototherapy for the treatment of hyperbilirubinemia. For instance, because the reflective layer 132 is relatively reflective of visible light, a greater amount of the light emitted by the phototherapy light source 116 is directed toward the child B. In addition, due to the relatively low level of heat reflectivity and the porosity of the reflective layer 132, relatively little heat is transmitted to the child B. As a result, phototherapy sessions using the reflective crib liner 110 may be conducted over greater durations relative to phototherapy sessions using some conventional phototherapy methods and equipment. Improvement in the efficacy of phototherapy may be realized.

It will be understood by those who practice the embodiments described herein and those skilled in the art that various modifications and improvements may be made without departing from the spirit and scope of the disclosed embodiments. The scope of protection afforded is to be determined solely by the claims and by the breadth of interpretation allowed by law.

What is claimed is:

1. A reflective crib liner for improving the efficacy of a phototherapy session performed using a bassinet comprising at least one wall defining an interior surface having a bottom portion, the reflective crib liner comprising:
a fabric layer having dimensions to accommodate placement of the reflective crib liner in the bassinet; and
a reflective layer formed so as to cover at least a portion of the interior surface, the reflective layer having a first degree of reflectance with respect to visible light and a second degree of reflectance with respect to heat that is substantially lower than the first degree of reflectance, the reflective layer comprising a porous material having a light diffusing surface.

2. The reflective crib liner of claim 1, further comprising a layer of padding material located proximate an undersurface of the reflective layer.

3. The reflective crib liner of claim 2, wherein the padding material is formed from a woven material.

4. The reflective crib liner of claim 1, wherein the reflective layer is formed so as to substantially cover the bottom portion of the interior surface of the bassinet.

5. The reflective crib liner of claim 1, wherein the reflective layer is formed from one of a woven material and a non-woven material.

6. The reflective crib liner of claim 5, wherein the reflective layer is formed from at least one of a reflective film and a woven material having reflective threads.

7. The reflective crib liner of claim 6, wherein the woven material comprises a metallic thread as one of a warp and a woof of the material.

8. The reflective crib liner of claim 7, wherein the woven material comprises a lamé material.

9. The reflective crib liner of claim 1, wherein the reflective layer is formed from a quilted material.

10. The reflective crib liner of claim 1, further comprising an elastic material fastened to at least one of the fabric layer and the reflective layer.

11. A phototherapy system comprising:
a bassinet comprising at least one wall defining an interior surface having a bottom portion;
a phototherapy light source mounted over the bassinet; and
a reflective crib liner located in the bassinet, the reflective crib liner comprising a fabric layer and a reflective layer formed so as to cover at least a portion of the interior surface of the bassinet, the reflective layer having a first degree of reflectance with respect to visible light and a second degree of reflectance with respect to heat that is substantially lower than the first degree of reflectance, the reflective layer comprising a porous material having a light diffusing surface.

12. The phototherapy system of claim 11, wherein the phototherapy light source is constructed and arranged to emit visible light having a wavelength range of 400-500 nm toward the bassinet.

13. The phototherapy system of claim 11, further comprising a layer of padding material located proximate an undersurface of the reflective layer.

14. The phototherapy system of claim 13, wherein the padding material is formed from one of a woven material.

15. The phototherapy system of claim 11, wherein the reflective layer is formed so as to substantially cover the bottom portion of the interior surface of the bassinet.

16. The phototherapy system of claim 11, wherein the reflective layer is formed from one of a woven material and a non-woven material.

17. The phototherapy system of claim 16, wherein the reflective layer is formed from at least one of a reflective film and a woven material having reflective threads.

18. The phototherapy system of claim 17, wherein the woven material comprises a metallic thread as one of a warp and a woof of the material.

19. The phototherapy system of claim 18, wherein the woven material comprises a lamé material.

20. The phototherapy system of claim 11, wherein the reflective layer is formed from a quilted material.

21. The phototherapy system of claim 11, further comprising an elastic material fastened to at least one of the fabric layer and the reflective layer.
